(19) United States
(12) Patent Application Publication
(10) Pub. No.: US 2008/0206380 A1
(43) Pub. Date: Aug. 28, 2008

(54) Preventing Agent Against Drug-Resistant Bacterial Infection

(76) Inventors: Kazunari Ushida, Nishinomiya-shi (JP); Takamitsu Tsukahara, Kawanishi-shi (JP); Yuri Sakai, Jyoyo-shi (JP); Wakoto Bukawa, Saitama-shi (JP); Noritaka Matsubara, Saitama-shi (JP)

Correspondence Address: MORGAN & FINNEGAN, L.L.P., 3 WORLD FINANCIAL CENTER, NEW YORK, NY, 10281-2101, US

(21) Appl. No.: 12/048,899

(22) Filed: Mar. 14, 2008

Related U.S. Application Data
(62) Division of application No. 11/083,513, filed on Mar. 18, 2005.

(30) Foreign Application Priority Data
Sep. 24, 2004 (JP) 2004-278572

(51) Int. Cl. A61K 35/74; A61P 31/04

(52) U.S. Cl. 424/780

(57) ABSTRACT

The present invention is to provide an agent for preventing and treating drug-resistant bacterial infection for livestock/fowls or fish and shellfish, using particular microbial agents as active components, without using synthetic antibacterial substances or antibiotics, and a method for preventing and treating its infection. By using lactic acid bacteria, their dead bacteria or treated substances thereof, or Mygasphaera elsdenii as active components, for an agent for preventing and treating drug-resistant bacterial infection for livestock/fowls or fish and shellfish carrying or being infected by drug-resistant bacteria such as Vancomycin, particularly by using Enterococcus faecalis, Enterococcus faecium as lactic acid bacteria, the above mentioned object was resolved.

### CROSS-REFERENCE TO RELATED APPLICATIONS

This application is a divisional under 35 U.S.C. §120 of prior U.S. patent application Ser. No. 11/083,513 filed on Mar. 18, 2005 which itself claims the foreign priority benefits under 35 U.S.C. §119(a)-(d) of Japanese Patent Application No. 2004-278572, filed Sep. 24, 2004, both of which are incorporated herein by reference in their entirety.

### TECHNICAL FIELD

The present invention relates to an agent for preventing and treating infection for livestock/fowls or fish and shellfish against drug-resistant bacteria such as Vancomycin-resistant Enterococci (also referred as VRE, abbreviated) or multidrug-resistant bacteria of livestock/fowls or fish and shellfish, containing lactic acid bacteria, their dead bacteria (non-alive bacteria) or treated substances thereof, or Megasphaera elsdenii as active components, and to a method for preventing and treating the infection thereof.

### BACKGROUND ART

Recently, as the human population ages and medical services progress, it happens that opportunistic infections of Vancomycin-resistant Enterococci (VRE) Methicillin- or Vancomycin-resistant Staphylococcus aureus (MRSA or VRSA), or enteropathogenic Escherichia coli having multidrug-resistant ability, which are not serious for healthy people, occur in medical institutions These pathogens can be hardly treated with antibacterial substances, which is causing a serious problem. As one of the reasons of the drug resistance, it has been pointed out that heavy usage of antibacterial substances in livestock farms and aquafarms induce selection of drug-resistant bacteria, which are transmitted to human via animal and sea food products, thus affecting human medical care. This problem has been discussed not only in Japan but also all around the world.

Conventionally, various synthetic antibacterial agents were known as antibacterial agents against these kind of resistant bacteria. Examples including quinoline carboxylic acid derivative and its salt (see e.g. Japanese Laid-Open Patent Application No. 6-73056); new macrolide compounds being antibiotics (see e.g. Japanese Laid-Open Patent Application No. 2001-238692) are known. Moreover, following examples containing ingredient derived from natural product as main constituent are known: for example extract of the pileus part of the fruit body of varnished conks or the like, (e.g. Japanese Laid-Open Patent Application No. 2000-143529); germicides for Vancomycin-resistant Enterococci containing Hinokitiol, its metal complex, or their salt as active components (see e.g. Japanese Laid-Open Patent Application No. 2001-131061); anti-disease feed additives containing enzyme-treated substances wherein quercetin content is increased by adding water to Fagopyrum tataricum Gaertn. to induce self-enzyme treatment of the same (see e.g. Japanese Laid-Open Patent Application No. 2001-292706). Further, as for the art related to lactic acid bacteria, preventing agent against infection containing microbial bacteria belonging to Enterococcus or treated substances thereof such as unltrasonic crushed substances as active components (see e.g. Japanese Laid-Open Patent Application No. 8-283166); Lactobacillus caseiproducing antibacterial substances showing growth inhibiting effect and toxicity reducing effect to microorganisms (see e.g. Japanese Laid-Open Patent Application No. 2001-333766); phenyllacetic acidproducedby using lactic acid bacteria, wherein the lactic acid bacteria is Enterococcus faecalis (see e.g. Japanese Laid-Open Patent Application No. 2000-300284), are known.

On the other hand, no drugs containing lactic acid bacteria, their dead bacteria or treated substances thereof as an active component were known as agent for preventing and treating drug-resistant bacterial infection for livestock/fowls or fish and shellfish.

### DISCLOSURE OF THE INVENTION

It is reported that the amount used of antibacterial substances is 520 t/year for human recently, while 1060 t and 230 t are used for drugs for animals and for feed additives, respectively, which makes a total of 1290 t. By simple comparison with the amount used for human, almost two-fold amount is used for animal. Actually, according to a nationwide research on the actual situation of the sensitivity of antibacterial substances of bacteria derived from domestic animals, carried out under the cooperation of the country, prefectures and the like, it has been suggested that the proportion of drug-resistant bacteria to antibacterial substances increases proportionally to the amount used of the antibacterial substances.

While drug-resistant bacteria are becoming a big problem, interested persons including drug manufacturers have no objections to keep the amount of antibacterial substances to be used in livestock farms and aquafarms at the minimum necessary, and to reduce as much as possible the dosage under appropriate usage. The Ministry of Agriculture, Forestry and Fisheries in Japan is now consulting to the food safety committee, and among the currently designated 29 components of antibacterial feed additives, they are considering to cancel the designation of 4 components which are not planned to be produced from now on, to review the designation according to scientific estimation for the 9 components similar to human drugs, and to continue the designation for the 16 components specific to domestic animals. On the other hand, as antibacterial drugs for animals are essential for treating animal diseases, it is considered to authorize continuously its use in principle, assuming the appropriate usage of the minimum necessary based on the diagnosis of a veterinarian.

When the same antibacterial substance is used for the treatment of pneumonia or diarrhea for a long period of time, sometimes it happens that bacteria being resistive to the drug appear and that the disease cannot be cured easily. As for antibacterial substances, there are antibiotics and synthetic antibacterial agents. Antibiotics are defined as follows by Waksman in 1942: “a substance produced byamicroorganism, being a chemical substance having the ability of inhibiting the growth of the other microorganism (particularly pathogenic microorganisms)”. On the other hand, synthetic antibacterial agents are antibacterial substances synthesized chemically. Many antibacterial substances are now synthesized (semi-synthesized) chemically from substances produced by microorganisms. However, these are classified as antibiotics. Generally, the term of “antibacterial substances for animals” is used as a combination of (1) antibacterial agents (drugs) for animals having as object the treatment of disease, and (2) antibacterial feed additives (antibacterial substances promoting growth, that are not drugs) to be added to feed, at a low concentration for a long period of time, in order to “promote growth” or “ameliorate feed efficiency” of edible animals.

Drug-resistant bacteria relate to bacteria showing resistance to antibacterial substances. When a disease is developed due to drug-resistant bacterial infection, even when an antibacterial substance is used for treatment, the disease is not cured or needs a long time for a complete cure. As for Salmonella typhimurium that can be the source of human food poisoning induces a disease also when affecting animals including domestic animals, multidrug-resistant bacteria (bacteria showing resistance to various bacteria) named DT104 is being a problem. Moreover, as for Campylobactor being offending bacteria of food poisoning, resistant bacteria to antibacterial substances used for treatment of human such as Fluoroquinolone (that is, new quinolones) are being a problem. When domestic animals are infected by Campylobactor, almost no symptoms are shown. Moreover, resistant bacteria do not always affect everyone. Most of offending bacteria which are being a problem among human drug-resistant bacteria, are indigenous bacteria in dermis, tonsil or intestinal tracts and do not have influence on healthy people. However, person whose immunity are decreased due to diseases or the like, they may become sick by opportunistic or hospital infection. There are bacteria inducing serious problems, such as MRSA or VRE. MRSA or VRE also infect domestic animals, but do not induce diseases of domestic animals. In this manner, resistant bacteria being a big problem, are serious for human disease, but not always induce disease of domestic animals. Moreover, unlike pathogen, it is difficult to estimate the existence of resistant bacteria, by just looking the farm.

When animals develop disease caused by bacteria, antibacterial substances are used for treating the disease. At that time, a part of bacteria obtain resistance, sensitive bacteria are killed by antibacterial substances, and only resistant bacteria survive. In that manner, drug-resistant bacteria increase when various antibiotics are used to human or domestic animals. Mutation during bacterial proliferation, induction of resistant genes that other bacteria have, or the like can be exemplified as trigger for bacteria to obtain drug resistance. Various resistant genes became apparent up to now. For example, some types of genes related to tetracycline resistance are known, and the resistance to one agent is not always caused by particularly limited to a single resistant gene.

To show resistance to antibiotics, bacteria have to inactivate agents surrounding the bacteria, or to prevent drugs from reaching the site of bacteria where the drug become active. In order to inactivate the agent, resistant bacteria produce enzyme (inactivated enzyme) that degrade or modify the agent. In order to prevent the agent from reaching the site of action, the mechanisms to prevent the invasion of the drugs into the bacteria (decrease of permeability of the drugs of the cytoplasmic membrane of bacteria), to modify the structure of the site where the drugs become active (change of primary site of action of the drugs), and to exclude the agent having invaded into the bacteria outside the bacteria (drug excluding pump), are related.

As for drug-resistance, in some case, the resistance does not contain congenitally the site of action of the drug as it is the case for spontaneous resistance. In other case, the resistance is generated by obtaining resistant gene posteriori. Moreover, as for resistant genes, there are genes being transmitted from resistant bacteria to sensitive bacteria, and genes that are not transmitted. In the resistant mechanism preventing the agent from reaching the site of action, drug resistance is rarely transmitted to other bacteria, while in the resistant mechanism producing enzyme that inactivates drugs, resistance may be transferred via plasmid or genes such as tranpozon. Resistant bacteria having thus obtained resistance can change other bacteria to resistant bacteria. Therefore, resistance of bacteria that does not induce diseases may be transferred to pathogens. Particularly, the resistant mechanism for VRE has been well investigated and it is known that the resistant mechanism van A, B of VRE are generated when are cell walls and pentapeptide of peptidoglycan murein are substituted to D-alanyl-D-lactate.

Moreover, there are other problems such as cross-resistance and coresistance. Cross-resistance is a phenomenon showing resistance to antibacterial susbstances of similar type, while coresistance is a resistant mechanism that has obtained resistance to a number of different types of agents at once. The bacteria having obtained resistance to antibacterial substances by that mechanism show resistance to drugs that have been used in the past.

Appearance of drug-resistant bacteria is deeply related with the use of drugs. Resistant bacteria increase according to the increase of the amount of used drugs. From the recent results of test of drug sensitivity resistance which have been reported up to now, many resistant bacteria against drugs that are used from a long time ago and have been highly consumed in Japan, have been found. Usually, the number of resistant bacteria decreases when the drug is not used any more. From a Danish research, it has been clarified that after the use of antibacterial feed additives has been stopped, the number of drug-resistant bacteria decrease. However, in an investigation performed 7 years after the use of antibacterial feed additives has been stopped, resistant bacteria against the eliminated drug have been found, though in a small rate. Therefore, when it is selected by a drug for some reason, there is still a remaining risk that resistant bacteria increase.

The problem of resistant bacteria has been pointed out in the 1990s as a worldwide issue, having a risk that “if antibacterial substances are used for animals, increase of human resistant bacteria will be induced, and treatment of human diseases will become difficult”. Therefore, WHO (World Health Organization) organized conferences to study this issue by exparts (1997: Berlin; 1998: Geneva). In these international conferences, the importance of monitoring to understand the situation of how the drug-resistant bacteria is distributed and spread between animals and human (trend survey and information gathering on resistant bacteria), has been pointed out. Then, OIE (World Organization for Animal Health) established a guideline for drug-resistant bacteria in 2000, to integrate the method of survey of drug-resistant bacteria performed in each country, that have been enacted on May 2003. Moreover, in the joint conference of FAO/OIE/WHO held in December 2003, it was decided that efforts should be made to decrease the risk of resistant bacteria, as “the risk of resistant bacteria of edible animals to human health cannot be denied”. There, the need of investigating also the trend of appearance of resistant bacteria has been pointed out, as there is a risk that resistant bacteria may appear in animals other than domestic animals, due to use of drug for pets or in aquaculture industry, or to use of antibacterial substances as pesticides. Thus, it can be understood that the problem of drug-resistant bacteria is a mainstream in the international community.

To prevent appearance of resistant bacteria in the field of animal industry, there may be methods for prohibiting or limiting the use of antibacterial substances for animals. However, antibacterial substances play an important role in producing cheap and safe animal products in a stable manner. Moreover, without antibacterial substances, it will be impossible to treat animals suffering from diseases. Therefore, it is difficult to prohibit all the antibacterial substances. On the other hand, responding to the voice from food industry asking for safe animal products without drug residue, there are farms among broiler producers that are making brands such as chemical-free chickens and limiting voluntarily the use of antibacterial substances. The consumers are highly concerned with the food safety/security recently, and the problem of drug-resistant bacteria and that of the residue of antibacterial substances and the like in food products are inextricably linked. Therefore, when using antibacterial substances, it is important to restrict the use of effective drug at the minimum necessary, according to diagnosis or test results before using.

The object of the present invention is to provide a safe agent for preventing and treating drug-resistant bacterial infection against drug-resistant bacterial infection of livestock/fowls or fish and shellfish, that do not use synthetic antibacterial agents or antibiotics, and thus to contribute for preventing drug-resistant bacterial infection in human.

The present inventors worried about the drug-resistant bacterial infection from livestock/fowls to human, and made a keen study. There, even when antibiotic Avoparcin (AVP) was not added to Japanese pigs, the possibility of Vancomycin-resistant bacteria (VRE) to turn out positive was suggested. It was thought that colonization of VRE in domestic animals was mainly attributed to AVP, but there may be other reasons in Japan. Therefore, the present inventors carried out experimental infection test of human-derived VRE using chicken as a bird model, and investigated the possibility of propagation transmitted from external factors, which was confirmed from the results. They carried out further experiments, and tried to prevent and treat drug-resistant bacteria from livestock/fowls or fish and shellfish carrying bacteria or being infected by drug-resistant bacteria such as Vancomycin. They have found that drugs containing lactic acid bacteria, their dead bacteria or treated substances thereof, or Clostridium butyricum using lactic acid, i.e. Megasphaera elsdenii as active components, show notable effects as a preventing and treating agent. Thus, they have completed the present invention.

In other words, the present invention relates to: an agent for preventing and treating drug-resistant bacterial infection for livestock/fowls or fish and shellfish, containing lactic acid bacteria, their dead bacteria or treated substance thereof, or Megasphaera elsdenii as active components; the agent for preventing and treating drug-resistant bacterial infection for livestock/fowls or fish and shellfish, wherein the lactic acid bacteria are bacteria belonging to Enterococcus; the agent for preventing and treating drug-resistant bacterial infection for livestock/fowls or fish and shellfish, wherein the bacteria belonging to Enterococcus are Enterococcus faecalis; the agent for preventing and treating drug-resistant bacterial infection, wherein Enterococcus faecalis is EC-12 (IFO 16803); the agent for preventing and treating drug-resistant bacterial infection for livestock/fowls or fish and shellfish, wherein EC-12 (IFO 16803) is their dead bacteria; the agent for preventing and treating drug-resistant bacterial infection for livestock/fowls or fish and shellfish, wherein the bacteria belonging to Enterococcus are Enterococcus faecium.

Moreover, the present invention relates to the agent for preventing and treating drug-resistant bacterial infection, wherein the lactic acid bacteria are bacteria belonging to Lactobacillus; the agent for preventing and treating drug-resistant bacterial infection, wherein the lactic acid bacteria are lactic acid bacteria derived from host animals; the agent for preventing and treating drug-resistant bacterial infection for livestock/fowls or fish and shellfish, wherein the drug-resistant bacteria are Vancomycin-resistant Enterococcus (VRE) or multidrug-resistant bacteria; the agent for preventing and treating drug-resistant bacterial infection for livestock/fowls or fish and shellfish, wherein the dead bacteria are dead bacteria being heat treated; a method for preventing and treating drug-resistant bacterial infection of livestock/fowls or fish and shellfish by administering orally composition containing lactic acid bacteria, their dead bacteria or treated substances thereof, or Megasphaera elsdenii as active components to livestock/fowls or fish and shellfish; the method for preventing and treating drug-resistant bacterial infection of livestock/fowls or fish and shellfish, wherein lactic are bacteria belonging to Enterococcus; the method for preventing and treating drug-resistant bacterial infection of livestock/fowls or fish and shellfish, wherein the bacteria belonging to Enterococcus are Enterococcus faecalis.

Furthermore, the present invention relates to the method for preventing and treating drug-resistant bacterial infection for livestock/fowls or fish and shellfish, wherein Enterococcus faecalis is EC-12 (IFO 16803); the method for preventing and treating drug-resistant bacterial infection of livestock/fowls or fish and shellfish, wherein EC-12 (IFO 16803) is their dead bacteria; the method for preventing and treating drug-resistant bacterial infection of livestock/fowls or fish and shellfish, wherein the bacteria belonging to Enterococcus are Enterococcus faecium; the method for preventing and treating drug-resistant bacterial infection of livestock/fowls or fish and shellfish, wherein the lactic acid bacteria are bacteria belonging to Lactobacillus; the method for preventing and treating drug-resistant bacterial infection of livestock/fowls or fish and shellfish, wherein the lactic acid bacteria are bacteria derived from host animals; the method for preventing and treating drug-resistant bacterial infection of livestock/fowls or fish and shellfish, wherein the drug-resistant bacteria are Vancomycin-resistant Enterococcus (VRE) or multidrug-resistant bacteria; the method for preventing and treating drug-resistant bacterial infection of livestock/fowls or fish and shellfish, wherein the dead bacteria are dead bacteria being heat treated.

### BRIEF EXPLANATION OF DRAWINGS

FIG. 1 is a figure that shows the change of VRE positive rate by the administration of each bacteria strain of the present invention.

### BEST MODE OF CARRYING OUT THE INVENTION

As for the agent for preventing and treating drug-resistant bacterial infection for livestock/fowls or fish and shellfish of the present invention, there is no specific limitations as long as it contains lactic acid bacteria, their dead bacteria or treated substances thereof, or Megasphaera elsdenii as active components. Moreover, as for the method for preventing and treating drug-resistant bacterial infection of livestock/fowls or fish and shellfish of the present invention, there is no specific limitation as long as it is a method for administering orally agents containing lactic acid bacteria, their dead bacteria or treated substances thereof, or Megasphaera elsdenii as active components to livestock/fowls or fish and shellfish. The above-mentioned agent for preventing and treating drug-resistant bacterial infection can be used directly, or in any forms including dosage forms.

As for Lactococcus used in the present invention, examples include the following: Enterococcus faecalis, Enterococcus faecium, Lactococcus lactis, Lactococcus plantarum, Lactococcus raffinolactis, Streptococcus thermophilus, Leuconostoc lactis, Leuconostoc mesenteroides, Pediococcus. As for Lactobacillus used in the present invention, examples include the following: Lactobacillus acidophilus, Lactobacillus salivarius, Lacobacillus brevis, Lactobacillus rhamnosus, Lactobacillus plantarum, Lactobacillus helveticus, Lactobacillus fermentum, Lactobacillus paracasei, Lactobacillus casei, Lactobacillus delbrueckii, Lactobacillus reuteri, Lactobacillus gasseri, Lactobacillus johnsonii, Lactobacillus kefiri, and Lactobacillus buchneri. Moreover, as for Bifidobacterium, examples include the following: Bifidobacterium breve, Bifidobacterium animalis, Bifidobacterium bifidum, Bifidobacterium infantis, Bifidobacterium longum, Bifidobacterium pseudolongum, Bifidobacterium thermophilum, and Bifidobacteriuum adolecentis.

Moreover, as for lactic acid bacteria used in the present invention, lactic acid bacteria derived from host animals can be preferably exemplified. The lactic acid bacteria derived from host animals is thought to colonize in intestinal tract before drug-resistant bacteria such as VRE and inhibit the colonization of drug-resistant bacteria afterward. On the contrary, it is thought that dead bacteria such as Enterococcus faecalis promote generation of IgA, IgG specific to VRE and the like being related species of Enterococcus faecalis, or antibacterial substances such as lysozyme or defensin, having strong germicidal effect to Gram positive bacteria, and prevent infection of VRE and the like. Furthermore, Megasphaera elsdenii that produce butyric acid from lactic acid, can be used for preventing drug-resistant bacterial infection. Megasphaera elsdenii can be isolated for example from pig colon.

These lactic acid bacteria and the like can be used by compounding one or two or more bacteria species. These lactic acid bacteria and the like can be obtained by culturing under any condition according to a commonly known method.

Among the above-mentioned lactic acid bacteria, microorganisms belonging to Enterococcus faecalis such as Enterococcus faecalis ATCC19433, Enterococcus faecalis EC-12 can be preferably exemplified. Especially, Enterococcus faecalis EC-12 (IF016803) is more preferable. 16SrDNA of Enterococcus faecalis EC-12 (IFO 16803) is registred as “AB15482” at the National Institute of Genetics. Enterococcus faecalis EC-12 has been deposited with the International Patent Organism Depository of National Institute of Advanced Industrial Science and Technology (AIST Tsukuba Central 6, 1-1, Higashi 1-chome Tsukuba-shi, Ibaraki-ken, 305-8566, Japan) on Feb. 25, 2005 under the provisions of the Budapest Treaty on the International Recognition of the Deposit of Microorganisms for the Purposes of Patent Procedures, and assigned accession number FERMABP-10284, incorporated herein by reference.

The bacterial characteristics of Enterococcus feacalis EC-12 used in the present invention are shown in Table 1. As for the method for culturing the Enterococcus feacalis EC-12, there is no specific limitation including the commonly known method for culturing lactic acid bacteria. However, examples include a culture by using a medium for growth of lactic acid bacteria, maintaining the culture pH near neutral point at 37° C., for 5-120 hours, preferably for 16-28 hours, and to obtain culture solution containing about 10⁷ to 10¹⁰/ml, preferably 10⁸ to 10¹⁰/ml of live bacteria.

TABLE 1

| | |
|---|---|
| Deposit Number | IFO16803 |
| strain | E. faecalis EC-12 strain |
| shape of cells | globular |
| gram staining property | + |
| catalase | − |
| NaCl (6.5%) proliferation | + |
| proliferation in a pH 9.6 medium | + |
| proliferation in a bile acid medium (4%) | + |
| arabinose | − |
| melibiose | − |
| sorbose | − |
| melezitose | + |
| sorbitol | + |

In the present invention, it is preferable to use live bacteria, their dead bacteria or treated substances thereof for lactic acid bacteria, and live bacteria for Megasphaera elsdenii. As for the above-mentioned dead bacteria, dead bacteria suspension or its dried material obtained by the following steps can be exemplified: culturing and harvesting bacteria of lactic acid bacteria by a common method, washing and dehydrating by centrifuge the bacteria; repeating the operation of washing and dehydration according to need, and suspending the resultants in distilled water, normal saline solution or the like; heating the suspension at 80-115° C. for 30 min to 3 sec. Other examples include dead bacteria suspension or its dried material obtained by irradiating gamma ray or neutron radiation to the above-mentioned dead bacteria suspension. The drying means of the dead bacteria suspension is not specifically limited as long as it is a commonly known drying means, and spray drying or lypholizing can be exemplified. Treatment with enzyme, surfactant, or by grinding and crushing can be performed before and after sterilization treatment by heating and the like, or before and after drying treatment depending on circumstances. The resultants of these treatments are also within the scope of dead bacteria or treated substances thereof of the present invention.

When using the above-mentioned agent for preventing and treating drug-resistant bacteria or its components as dosage forms, it can be compounded with additives such as carrier including starch, lactose, soy protein; excipient, binding agent, disintegrator, lubricant, stabilizer, suspending agent and the like to make dosage forms in form of powder, tablet, granules, capsules, liquid or the like, according to common procedures. Moreover, when it is combined with prebiotech material such as gluconate; oligosaccharides including galacto oligosaccharide, fructo oligosaccharide; or dietary fiber material including cellulose, β-glucan, or chitosan, it is more preferable as synergistic effects can be anticipated. The dosage forms can be directly administered, or fed by mixing to feeds or the like.

The agent for preventing and treating of the present invention had exhibited antibacterial effect particularly to VRE, more particularly to VRE being standard strain of Enterococcus faecalis derived from human. Therefore, the agent for preventing and treating of the present invention is widely applied to Enterococcus being heat resistant, or salt tolerant.

As for livestock/fowls being the target of prevention and treatment of drug-resistant bacteria of the present invention to domestic animals and so on, livestock including cattle, pig, horse, sheep, goat; or fowls including chicken, duck, ostrich can be exemplified. It can be applied to livestock/fowls of any age in days, or years including lactation period or feeding period. Particularly, baby pigs before and after weaning period, or chicks have weak power of resistance as intestinal bacterial flora are not yet matured and thus can be easily affected by VRE. Furthermore, as for fish and shellfish, fish and shellfish generally cultivated such as yellow tail, amberjack, flatfish, read sea bream, eel, prawn, clam can be preferably exemplified.

As for the forms of administering lactic acid bacteria, their dead bacteria or treated substances thereof, or Megasphaera elsdenii of the present invention to livestock and the like, methods for administering orally directly to domestic animals, or method for feeding by mixing them to feeds or drinking water can be exemplified, and any one of these can be used. At that time, it is more preferable when it is combined with prebiotech materials such as sodium gluconate; oligosaccharides including galacto oligosaccharide, fructo oligosaccharide; or dietary fiber material including cellulose, β-glucan, or chitosan, as synergistic effects can be anticipated.

The dose or number of times to administer the agent for preventing and treating drug-resistant bacterial infection of the present invention or by its method, can be appropriately determined according to the types of livestock/fowls, body weight, age in days or in months, pathology or recovering condition. For example, when dead bacteria or treated substance thereof of Enterococcus faecalis EC-12 are used for chickens, chicks, the dose can be mixed into feed so that the administering rate become 0.0001-0.05% of feed for chicks, to administer the usual feeding amount by the usual number of times of feeding per day. Moreover, as for pigs, it can be added in an amount of 0.0001% to 0.05% to baby pigs, particularly before and after weaning period.

### EXAMPLES

The present invention will be explained in reference with the examples in the following. However, the technical scope of the present invention is not limited to these.

### Example 1

### Preparation of Dead Bacteria of Enterococcus faecalis EC-12

Enterococcus faecalis EC-12 (IFO 16803) was cultured in a Rogosa medium at 37° C. for 24 hours. The culture solution was inoculated in an amount of 0.1 (v/v) % to a liquid medium containing 4% yeast extract, 3% polypepton, and 10% lactose. By adjusting the pH to 6.8-7.0 by using sodium hydroxide with a pH stat, neutralizing culture was performed at 37° C. for 22-24 hours.

After the culture has completed, the bacteria were separated with a continuous centrifuge and collected. Then, water was added to dilute up to the original liquid level, and the bacteria were separated again with a continuous centrifuge, and collected. This operation was carried out 4 times to wash the bacteria. Then, the washed bacteria were suspended in an appropriate water level, sterilized at 100° C. for 30 min, and dried by using a spray drier to prepare a heat treated bacteria powder.

### Example 2

### Infection Test of Human-Derived VRE

Bacterial culture of 2 VRE bacteria strain (2 strains of human-derived standard bacteria) was forcibly administered orally in an amount of about 10⁸/chick to 1 day-old VRE-free broiler chicks (2 groups, 6 chicks per group). At day 0.5, 1, 3, 7 and 14 after administration, fecal swabs were collected, and smeared to an EF agar medium supplemented with 10 μl/mL of Vancomycin (VCM) The resultant was cultured at 37° C. for 48 hours, the grown colony was collected, and was identified to belong to Enterococcus, from its Gram staining, morphology, and fermenting ability. At day 21 after administration, the chicks were dissected and the colonization to each gastrointestinal tracts including crop, stomach, small intestine and cecum was examined. As a result, VRE was isolated from all swabs of day 0.5 to 14 after administration, while at day 21 after administration, VRE was isolated from all gastrointestinal tracts including crop, stomach, small intestine and cecum. It has been clarified that VRE can be colonized at least for 21 days in broiler intestinal tract. The fact that human-derived VRE infect broiler chicks, suggests that VRE contamination in poultry housings can be induced by contamination from external living animals.

### Example 3

### Inhibition Effect of VRE Colonization in Intestinal Tract

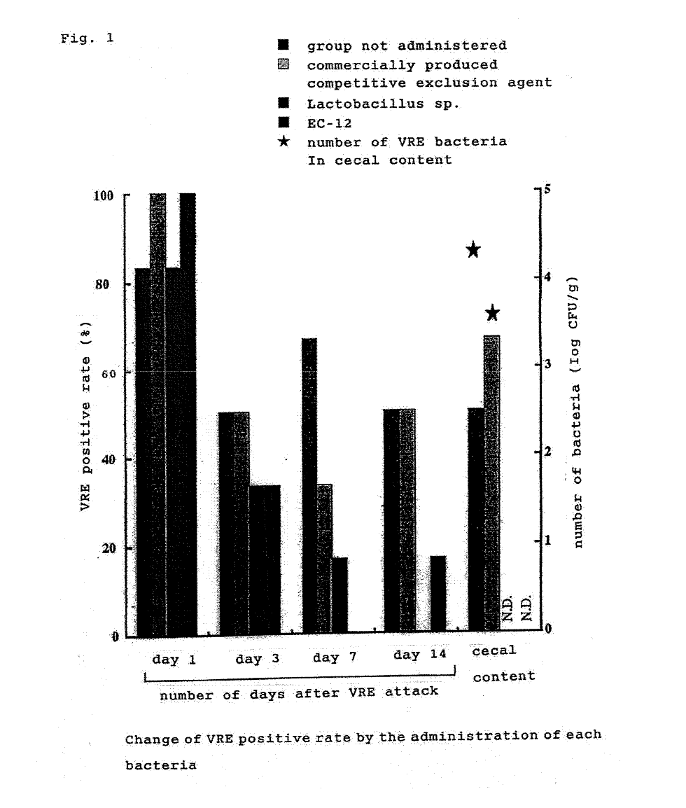

1 day-old VRE-free broiler chicks were used (4 groups, 6 chicks per group). Four groups were made as follows: control group not administered; group administered with dead lactic acid bacteria powder (EC-12)-added feed; group forcibly administered orally with chicken fecal-derived Lactobacillus sp.; and group spray-administered with commercially produced Aviguard (competitive exclusion agent; Bayer). Lactobacillus sp. was forcibly administered once at the time of 1 day-old. A competitive exclusion agent was administered by spraying at the time of 1 day-old. EC-12 was administered by adding to the basic feed, in an amount of 0.05% from the time of 1 day-old until the time of examination by dissection. VRE strain whose colonization to intestinal tract was confirmed in Example 2, was forcibly administered orally to all of 2 days-old chicks. At days 1, 3, 7 and 14 after VRE attack (infection), fecal swabs were collected and the bacterial discharge condition of VRE was estimated qualitatively in the same manner as Example 2. Chicks were dissected and examined at day 14 after VRE attack (infection), and the number of VRE bacteria in cecal content was determined. The results are shown in FIG. 1. As it is shown in FIGS. 1, 3 of 6 chicks in the control group not administered turned out Positive until day 14 after VRE attack (infection), while all of the Lactobacillus sp.-administered group turned out negative, 1 chick of the EC-12-administered group turned out positive. 3 of 6 chicks in the competitive exclusion agent-administered group turned out positive. The number of bacteria in cecal content showed a similar trend, and the level of all chicks of the Lactobacillus sp.- or EC-12-administered group were below detection limit. Thus, it has been clarified that lactic acid bacteria such as Lactobacillus or EC-12 were useful for inhibiting VRE colonization.

### Example 4

### Inhibition Effect of VRE Colonization in Intestinal Tract

As for bacteria or its dosage form used in the present invention, dead EC-12 bacteria, Lactobacillus sp. and Enterococcus Sp. were used as lactic acid bacteria; butyric acid bacteria, Megasphaella elsdenii, isolated from pig large intestine were used as bacteria using lactic acid; and Lactobacillus sp. and Megasphaella elsdenii were used as mixed bacteria. As control substance, an antibiotic Aviguard (Bayer) was used. 24 broiler chicks (1 day-old, 12 males, 12 females) were used as test animals. As for basic feed, commercially available testing formula feed (testing standard feed for early stage broiler, SDB No. 1, Nippon Formula Feed Mfg. Co. Ltd.) was used. 1 day-old chicks were housed in a closed livestock barn and their body weight were measured. They were divided into 4 groups, so that the body weight were approximately even between groups, and were housed in a stainless steel cage per group. As for administration pattern, EC-12 and Enterococcus faecium were added to the feed and administered from the initiation of the test (1 day-old) until the termination of the test (16 days-old). Lactobacillus sp., Megasphaera elsdenii, and a mixture of Lactobacillus sp. and Megasphaera elsdenii were forcibly administered orally at the time of 1 day-old. Aviguard was administered once at the time of the initiation of the test (1 day-old) according to its use/dosage.

As for VRE infection, VRE was forcibly administered orally to 2 days-old chicks. As for the bacterial strain, pig-derived field isolated strain E6 strain was used, and bacterial culture (bacterial concentration 10⁸/0.5 mL) was administered by an amount of 0.5 mL each time. At the time of VRE administration, fecal swabs of all the chicks were collected and smeared to an EF agar medium supplemented with Vancomycin (VCM), to confirm in advance to be VRE free.

After the VRE administration, feces were collected with a sterilized cotton bud at day 0, 1, 3 and 7, and smeared to an EF agar medium supplemented with VCM to confirm the colonization or passage of VRE. The results are shown in Table 2. Further, at the time of the termination of the test (day 14 after Vancomycin administration), dissection was carried out. Cecum was extracted, and 10-fold serial dilution was performed using the sample diluent. Diluent of an adequate stage was smeared to an EF agar plate supplemented with VCM and LBS agar plate. The number of VCR bacteria in cecal content and its results are shown in Table 2.

TABLE 2

| | | | spray- | | | | | | |
|---|---|---|---|---|---|---|---|---|---|
| | | | admin- | administered | administered | | forcibly | Forcibly | |
| | | | istered | with EC-12 | orally with | Forcibly | administered | administered | |
| | | control | with | added feed | Enterococcus | administered | orally with | orally with | Kruskal- |
| | | not | Aviguard | (during | faecium during | orally with | Megasphaera | Lactobacillus sp. + | Wallis |
| day of | | admin- | (1-day | testing | testing period | Lactobacillus sp. | elsdenii | M. elsdenii | test |
| determination | | istered | old) | period) | (1-day old) | (1-day old) | (1 day-old) | (1 day-old) | p level |
| day 1 | number | 5 | 6 | 6 | 6 | 5 | 6 | 6 | 0.53 |
| after | being | | | | | | | | |
| attack | positive | | | | | | | | |
| | number | 1 | 0 | 0 | 0 | 1 | 0 | 0 | |
| | being | | | | | | | | |
| | negative | | | | | | | | |
| | positive | 83 | 100 | 100 | 100 | 83 | 100 | 100 | |
| | rate | | | | | | | | |
| day 3 | number | 3 | 3 | 2 | 3 | 2 | 2 | 1 | 0.92 |
| after | being | | | | | | | | |
| attack | positive | | | | | | | | |
| | number | 3 | 3 | 4 | 3 | 4 | 4 | 5 | |
| | being | | | | | | | | |
| | negative | | | | | | | | |
| | positive | 50 | 50 | 33 | 50 | 33 | 33 | 17 | |
| | rate | | | | | | | | |
| day 7 | number | 4 | 2 | 0 | 3 | 1 | 3 | 1 | 0.01 |
| after | being | | | | | | | | |
| attack | positive | | | | | | | | |
| | number | 2 | 4 | 6 | 3 | 5 | 3 | 5 | |
| | being | | | | | | | | |
| | negative | | | | | | | | |
| | positive | 67 | 33 | 0 | 50 | 17 | 50 | 17 | |
| | rate | | | | | | | | |
| | | ab | ab | a | ab | | ab | ab | |
| day 14 | number | 3 | 3 | 1 | 0 | 0 | 0 | 1 | 0.004 |
| after | being | | | | | | | | |
| attack | positive | | | | | | | | |
| | number | 3 | 3 | 5 | 6 | 6 | 6 | 5 | |
| | being | | | | | | | | |
| | negative | | | | | | | | |
| | positive | 50 | 50 | 17 | 0 | 0 | 0 | 17 | |
| | rate | | | | | | | | |
| | | ab | ab | ab | ab | a | a | ab | |
| the number | mean | 20267 | 3850 | N.C. | 912 | N.C. | 467 | 400 | (positive |
| of | level | | | | | | | | rate) |
| bacteria | standard | 34411 | 4234 | N.C. | 455 | N.C. | 306 | N.C. | 0.004 |
| in cecal | deviation | | | | | | | | |
| content | detective | 50 | 67 | 0 | 50 | 0 | 50 | 17 | |
| (CFU/g) | rate | | | | | | | | |
| | | ab | ab | a | ab | a | ab | ab | |

As it is shown in Table 2, lactic acid bacteria themselves (Enterococcus faecium, Lactobacillus sp.), dead bacteria of lactic acid bacteria (EC-12), Megasphaera elsdenii and a mixture of Lactobacillus sp. and Megasphaera elsdenii have an effect for inhibiting colonization to VRE-infected domestic fowls.

### Example 5

### Infection Test of Vancomycin-Resistant Bacteria

Enterococcus faecalis ATCC 51299 strain was used as Vancomycin-resistant bacteria. MIC levels to various antibiotics of Enterococcus faecalis ATCC51299 strain are as shown in Table 3.

TABLE 3

| | MIC levels after 16 | MIC levels after 24 |
|---|---|---|
| | hours of culture | hours of culture |
| Antibiotics | (μg/mL) | (μg/mL) |
| Vancomycin | 16 | 64 |
| Ampicillin | 0.5 | 0.5 |
| Tetracycline | 0.5 | 1 |
| Gentamicin | >512 | >512 |
| Streptomycin | >512 | >512 |
| Oxacillin | 32 | 64 |
| Bacitracin | 64 | 128 |
| Teicoplanin | 1 | 1 |
| Chloramphenicol | 64 | 128 |
| Erythromycin | 512 | >512 |

6 males and 7 females of 1 day-old VRE free broiler chicks were used for the control group not administered, 3 males and 3 females of the same for the group forcibly administered orally with chicken fecal-derived Lactobacillus sp., 7 males and 6 females for EC-12 group. Thus, three groups were made. EC-12 was administered by adding to the basic feed in an amount of 0.05% dead-lactic acid bacteria powder, from the time of 1 day-old until the examination by dissection (during all the period). Chicken fecal-derived Lactobacillus sp. were forcibly administered orally at the time of 1 day-old. The mean body weight, and the mean body weight increased were determined for the three groups at days 1, 3, 7, and 14 after ATCC51299 strain attack (infection). The results are shown in Table 4.

TABLE 4
Mean body weight, mean body weight increased during testing period

| | | mean body weight (g) | | | | mean body weight increased (g) | | | | |
|---|---|---|---|---|---|---|---|---|---|---|
| | | time of | number of days after attack | | | number of days after attack | | | | |
| | | induction | with E. faecalis ATCC51299 | | | with E. faecalis ATCC51299 strain | | | | |
| tested group | | −1 | 0 | 7 | 14 | −1-7 | −1-14 | 7-14 | 0-7 | 0-14 |
| control not | mean level | 40.9 | 49.3 | 180.9 | 437.5 | 140.0 | 396.5 | 256.5 | 131.6 | 388.2 |
| administered | standard | 2.8 | 4.4 | 28.4 | 65.0 | 27.8 | 64.3 | 39.6 | 25.5 | 62.6 |
| | deviation | | | | | | | | | |
| forcibly and orally | mean level | 40.8 | 46.3 | 179.7 | 420.0 | 138.8 | 379.2 | 240.3 | 133.3 | 373.7 |
| administered with | standard | 2.5 | 2.3 | 20.2 | 45.6 | 21.5 | 46.2 | 40.4 | 18.6 | 45.2 |
| Lactobacillus sp. | deviation | | | | | | | | | |
| (1 day-old) | | | | | | | | | | |
| administered with | mean level | 40.8 | 49.7 | 196.2 | 466.2 | 155.4 | 425.5 | 270.1 | 146.5 | 416.5 |
| EC-12 added | standard | 2.6 | 3.9 | 23.8 | 56.8 | 22.7 | 55.6 | 37.0 | 21.1 | 54.2 |
| feed | deviation | | | | | | | | | |
| (during testing | | | | | | | | | | |
| period) | | | | | | | | | | |
| one-way analysis | | 0.99 | 0.21 | 0.24 | 0.24 | 0.23 | 0.23 | 0.30 | 0.23 | 0.25 |
| of variance | | | | | | | | | | |
| p level | | | | | | | | | | |

Next, fecal swabs of each of the three groups were collected at days 1, 3, 7, and 14 after ATCC51299 strain attack (infection), and the bacterial discharge condition of VRE was estimated. At day 14 after ATCC51299 strain attack (infection), examination by dissection was carried out and the number of VRE bacteria in cecal content was determined. Further, the translocation of ATCC51299 strain in blood, liver and spleen was examined. The results are shown in Table 5.

TABLE 5
Positive rate of E. faecalis ATCC51299 strain from feces, cecal content and each organ

| | | | | | | | number of bacteria | | | |
|---|---|---|---|---|---|---|---|---|---|---|
| | | | number of days after attack | | | | of E. faecalis | | | |
| | | time of | with E. faecalis ATCC51299 | | | | ATCC51299 strain | translocation | | |
| tested group | | induction | 0 | 1 | 3 | 7 | in cecal content | blood | liver | spleen |
| | | | | | | | 85,600 | | | |
| control not | number of | 0 | 0 | 100 | 100 | 77 | 100 | 0 | 0 | 0 |
| administered | bacteria | | | | | | | | | |
| | (CFU/g) | | | | | | | | | |
| | positive | (0/13) | (0/13) | (13/13) | (13/13) | (13/13) | (13/13) | (0/13) | (0/13) | (0/13) |
| | rate (%) | | | | | | | | | |
| | | | | | b | ab | b | | | |
| | | | | | | | 6,500 | | | |
| forcibly and orally | number of | 0 | 0 | 67 | 100 | 100 | 100 | 0 | 0 | 0 |
| administered with | bacteria | | | | | | | | | |
| Lactobacillus sp. | (CFU/g) | | | | | | | | | |
| (1 day-old) | positive | (0/6) | (0/6) | (4/6) | (4/6) | (4/6) | (6/6) | (0/6) | (0/6) | (0/6) |
| | rate (%) | | | | | | | | | |
| | | | | | b | b | b | | | |
| | | | | | | | 8,000 | | | |
| administered with | number of | 0 | 0 | 46 | 31 | 38 | 54 | 0 | 0 | 0 |
| EC-12 added feed | bacteria | | | | | | | | | |
| (during testing | (CFU/g) | | | | | | | | | |
| period) | positive | (0/13) | (0/13) | (6/13) | (6/13) | (6/13) | (7/13) | (0/13) | (0/13) | (0/13) |
| | rate (%) | | | | | | | | | |
| | | | | | | | a | | | |
| one-way analysis | | N.C. | N.C. | 0.42 | 0.006 | 0.01 | 0 | N.C. | N.C. | N.C. |
| of variance | | | | | | | | | | |
| p level | | | | | | | | | | |

Further, total IgA concentration in cecal content, and total IgG concentration in serum at the time of dissection for the above-mentioned three groups were determined by using ELISA. ELISA was determined with Chicken IgA ELISA Quantitation Kit (Bethyl Laboratories Inc., Montgomery, Tex.) and Chicken IgG ELISA Quantitation Kit (Bethyl Laboratories Inc.). The results are shown in Table 6.

TABLE 6
Total IgA concentration in cecal content and total IgG
concentration in serum at the time of dissection

| | | total IgA | total IgG |
|---|---|---|---|
| | | concentration | concentration |
| | | cecal content | serum |
| tested group | | [μg/ml] | [ng/ml] |
| control not | mean | 28.4 | 885 |
| administered | level | | |
| | standard | 5 | 287.2 |
| | deviation | | |
| | | B | ab |
| forcibly and | mean | 31.3 | 684.6 |
| orally | level | | |
| administered with | standard | 5.5 | 245.6 |
| Lactobacillus sp. | deviation | | |
| (1 day-old) | | AB | b |
| administered with | mean | 36.6 | 1127.9 |
| EC-12 added | level | | |
| feed | standard | 5.7 | 496.5 |
| (during testing | deviation | | |
| period) | | A | a |
| one-way analysis | | 0.002 | 0.06 |
| of variance | | | |
| p level | | | |

* significant difference between different codes (risk rate 5%)

Furthermore, serum of the above mentioned three groups were diluted to 20-fold, and IgG specific to membrane protein of ATCC51299 strain was determined similarly by ELISA at an absorbance of [490 nm]. The method was as follows. Membrane protein solution of VRE cytoplasma was diluted with a carbonate buffer [50 mM of Na₂CO₃, 50 mM of NaHCO₃, pH 9.6], and used as a coating antibody. Membrane protein solution of VRE cytoplasma was prepared as follows.

1) Enterococcus faecalis ATCC51299 was cultured in a GAM medium until MG stage.
2) The medium was centrifuged. Pellet was stirred with re-suspended solution [10 m of Mtris-Cl, 1 mM of MEDTA, 50 mM of NaCl, pH7.4], and sonicated on ice until cell walls were completely destroyed.
3) Sonicated solution was ultracentrifuged to remove intracellular fractions. [47000×g, 20 min, 4° C.; Hitachi himac CP65β (Hitachi Koki Co. Ltd.), Tokyo (Japan)].
4) Pellet of sonicated solution (membrane protein of VRE cytoplasma) was washed twice (stirred with re-suspended solution and untracentrifuged [47000×g, 20 min, 4° C.]).

Membrane protein pellet of VRE cytoplasma was stirred in a carbonate buffer, in order to become 10 mg/mL of membrane protein solution of cytoplasma. Protein solution was used for coating antibodies in VRE-specific ELISA assay. The results are shown in Table 7.

TABLE 7
IgG specific to E. faecalis ATCC51299 strain membrane
protein in serum at the time of dissection

| | | VRE specific IgG |
|---|---|---|
| Group tested | | [ng/mL] |
| control group not | mean value | 95.2 |
| administered | standard | 39.8 |
| | variation | |
| group forcibly | mean value | 142.7 |
| administered | standard | 76.3 |
| orally with | variation | |
| Lactobacillus sp. | | |
| (1 day old) | | |
| group | mean value | 190.7 |
| administered with | standard | 186.6 |
| EC-12 added feed | variation | |
| (during testing | | |
| period) | | |

As it is shown in Table 4, in the infection test of multidrug-resistant bacteria, as for the group forcibly administered orally with lactic acid bacteria of the present invention Lactobacillus sp. (1 day-old) and the group administered with EC-12 added feed (during all testing period), the mean body weight of group forcibly administered orally with lactic acid bacteria Lactobacillus sp. (1 day-old) was lower compared to that of the control group. However, as for the group administered with EC-12 added feed (during testing period), the body weight was about 6% higher at day 14 compared to that of the control group not administered.

Table 5 shows as for ATCC51299 strain bacteria in feces of day 7, that the positive rate was 77% for control group not administered, 100% for group forcibly administered orally with Lactobacillus sp. (1 day-old) 38% for group administered with EC-12 added feed (during testing period). It has been clarified that administration of EC-12 inhibits growth of bacteria of ATCC51299 strain, or significantly contributes to growth inhibition. Moreover, the mean number of bacteria of ATCC51299 strain in cecal content of positive living bodies, was 85600 for control group not administered, while it was 6500 for the group forcibly administered orally with Lactobacillus sp., and 8000 for the group administered with EC-12 added feed. The number of bacteria of ATCC 51299 strain in cecal content of the groups of the present invention was both significantly low compared to the control group.

Table 6 shows that the total IgA concentration [ng/ml] in 50-fold diluted cecal content of the groups of the present invention was both high, compared with that of the control group not administered. Moreover, as for the total IgG concentration [ng/ml], EC-12 administered group showed a much higher level compared with the control group not administered. Thus, EC-12 has an effect of enhancing immunity, and is effective to prevent diseases.

Table 7 shows that in the IgG specific to ATCC51299 strain membrane protein in serum at the time of dissection (20-fold diluted serum), EC-12 showed a very high level compared to that of the control group not administered. The results are similar with those shown in Table 6.

From the above-mentioned Example 5, lactic acid bacteria derived from host animals colonize in intestinal tract before drug-resistant bacteria such as VRE, and seem to inhibit colonization of drug-resistant bacteria afterward. On the contrary, it is thought that dead bacteria such as Enterococcus faecalis promote generation of IgA, IgG specific to VRE and the like, which are related species of Enterococcus faecalis and the like, and prevent infection of VRE and the like.

### INDUSTRIAL APPLICABILITY

By administering orally the agent for preventing and treating for livestock/fowls or fish and shellfish containing lactic acid bacteria, their dead bacteria or treated substances thereof, or Megasphaera elsdenii as active components of the present invention, the infection rate of drug-resistant bacteria decreases significantly in livestock/fowls or fish and shellfish. Therefore, without using antibiotics or synthetic antibacterial agents which were used conventionally for infections of livestock/fowls or fish and shellfish, the agent prevents and treats effectively infection of Vandomycin-resistant Enterococcus, or pathogens and the like having multidrug-resistant ability. Thus, prevention and treatment of such infection became possible.

1. A method for preventing and treating drug-resistant bacterial infection of livestock/fowls or fish and shellfish by administering orally composition containing lactic acid bacteria, their dead bacteria or treated substances thereof, or Megasphaera elsdenii as active components to livestock/fowls or fish and shellfish.

2. The method for preventing and treating drug-resistant bacterial infection of livestock/fowls or fish and shellfish according to claim 1, wherein the lactic acid bacteria are bacteria belonging to Enterococcus.

3. The method for preventing and treating drug-resistant bacterial infection of livestock/fowls or fish and shellfish according to claim 2, wherein the bacteria belonging to Enterococcus are Enterococcus faecalis.

4. The method for preventing and treating drug-resistant bacterial infection for livestock/fowls or fish and shellfish according to claim 3, wherein Enterococcus faecalis is EC-12 (IFO 16803).

5. The method for preventing and treating drug-resistant bacterial infection of livestock/fowls or fish and shellfish according to claim 4, wherein EC-12 (IFO 16803) is their dead bacteria.

6. The method for preventing and treating drug-resistant bacterial infection of livestock/fowls or fish and shellfish according to claim 2, wherein the bacteria belonging to Enterococcus are Enterococcus faecium.

7. The method for preventing and treating drug-resistant bacterial infection of livestock/fowls or fish and shellfish according to claim 1, wherein the lactic acid bacteria are bacteria belonging to Lactobacillus.

8. The method for preventing and treating drug-resistant bacterial infection of livestock/fowls or fish and shellfish according to claim 1, wherein the lactic acid bacteria are bacteria derived from host animals.

9. The method for preventing and treating drug-resistant bacterial infection of livestock/fowls or fish and shellfish according to claim 1, wherein the drug-resistant bacteria are Vancomycin-resistant Enterococcus (VRE) or multidrug-resistant bacteria.

10. The method for preventing and treating drug-resistant bacterial infection of livestock/fowls or fish and shellfish according to claim 1, wherein the dead bacteria are dead bacteria being heat treated.

11. The method for preventing and treating drug-resistant bacterial infection of livestock/fowls or fish and shellfish according to claim 2, wherein the drug-resistant bacteria are Vancomycin-resistant Enterococcus (VRE) or multidrug-resistant bacteria.

12. The method for preventing and treating drug-resistant bacterial infection of livestock/fowls or fish and shellfish according to claim 2, wherein the dead bacteria are dead bacteria being heat treated.
